(19) United States
(12) Patent Application Publication
(10) Pub. No.: US 2022/0160213 A1
(43) Pub. Date: May 26, 2022

(54) ENDOSCOPIC DEVICE WITH ADDITIONAL CHANNEL

(71) Applicant: UNITED STATES ENDOSCOPY GROUP, INC., Mentor, OH (US)

(72) Inventors: Alex Uspenski, Chardon, OH (US); John P. Winstanley, Madison, OH (US); Gary Mann, Painesville, OH (US); Scott Haack, Chardon, OH (US); Christopher J. Kaye, Berea, OH (US); Paul Martino, Solon, OH (US); Stephen T. Pastor, Sagamore Hills, OH (US); Reza Mohammadpour, Willoughby Hills, OH (US); Naji Chaar, Harrisburg, PA (US); Ryan Chambers, Bowling Green, OH (US); Tony Martella, Mentor, OH (US); Sven Huijs, Madison, OH (US); Victor Meza Hernandez, Cleveland Heights, OH (US); Keith R. John, Chardon, OH (US); Darren Gregan, Auburn Twp, OH (US); Kailey Vlack, Painesville, OH (US); Alison Streiff, Painesville, OH (US); Diego Jacho, Toledo, OH (US)

(21) Appl. No.: 17/533,300

(22) Filed: Nov. 23, 2021

Related U.S. Application Data
(60) Provisional application No. 63/117,286, filed on Nov. 23, 2020.

(51) Int. Cl. A61B 1/00; A61B 1/018; A61B 1/015

(52) U.S. Cl. CPC A61B 1/00137; A61B 1/00135; A61B 1/00087; A61B 1/00082; A61B 1/015; A61B 1/018

(57) ABSTRACT

An endoscopic device is described and includes at least an endoscope and one or more catheters, and a means for securing the endoscope to the catheter at a distal end of the endoscopic device. The means for securing the endoscope may be a sleeve or distal cap. The sleeve includes at least a first opening for securing the catheter therebetween and a second opening for securing the endoscope adjacent to the catheter.

### CROSS-REFERENCE TO RELATED APPLICATION

This application claims priority to and all benefit of U.S. Provisional Patent Application Ser. No. 63/117,286, filed on Nov. 23, 2020, the entire disclosure of which is incorporated herein by reference.

### TECHNICAL FIELD

The present disclosure relates generally to endoscopic devices and, more specifically, to a multichannel endoscopic device that allows for two or more tools to be used in concert during endoscopic procedures.

### BACKGROUND

Endoscopic submucosal dissection (ESD) is an endoscopic procedure done to remove tumors in, for example, the colon, stomach, or esophagus. These tumors are found in their early stages, as they have yet to reach the muscle layer where more significant damage could be done to the body. These early stage gastrointestinal tumors are removed using various tools and techniques. For example, an electrosurgical knife may be used in conjunction with a grasper of sorts to cut, retrieve, and remove a tumor. With many tools involved in a single ESD procedure, switching tools out during can be a slow, tedious process.

In view of the above limitations related to the use of multiple tools/devices during endoscopic procedures, there is an unmet need for an improved endoscopic device that allows for the use of two or more tools/devices in tandem during endoscopic procedures.

### SUMMARY

In one exemplary embodiment, an endoscopic device includes a catheter with a securing means at a distal end of the catheter and a mounting means at a proximal end of the catheter. The securing means is attached to the catheter at the distal end of the catheter and is configured to attach or otherwise secure the catheter to a first channel, which may include an endoscope or similar tool therein. The mounting means is attached to the catheter at the proximal end of the catheter and is configured to attach or otherwise secure the catheter to the first channel and/or a portion of a handheld endoscopic tool and/or similar accessory.

These and other objects, features, and advantages of the present disclosure will become apparent from the following detailed description of illustrative embodiments thereof, which is to be read in connection with the accompanying drawings.

### BRIEF DESCRIPTION OF THE DRAWINGS

These and other features of the present disclosure will become better understood with regard to the following description and accompanying drawings in which:

FIG. 1a illustrates an exemplary embodiment of an endoscope, in accordance with the general inventive concepts;

FIG. 1b illustrates an exemplary embodiment of an endoscopic device, in accordance with the general inventive concepts;

FIG. 2 illustrates another exemplary embodiment of an endoscopic device connected to an endoscopic system, in accordance with the general inventive concepts;

FIG. 3 illustrates an exemplary embodiment of one or more components of the endoscopic system of FIG. 2, in accordance with the general inventive concepts;

FIG. 4 illustrates a further exemplary embodiment of an endoscopic device, in accordance with the general inventive concepts;

FIG. 5 illustrates an exemplary embodiment of a means for securing an endoscope to embodiments of an endoscopic device, in accordance with the general inventive concepts;

FIG. 6 illustrates another exemplary embodiment of a means for securing an endoscope to embodiments of an endoscopic device, in accordance with the general inventive concepts;

FIG. 7 illustrates an exemplary embodiment of an endoscope adjacent to an additional channel, in accordance with the general inventive concepts;

FIG. 8a illustrates a perspective view of another exemplary embodiment of an endoscope in a first channel with trap door feature and adjacent to an additional channel, in accordance with the general inventive concepts;

FIG. 8b illustrates a second perspective view of the first channel with trap door feature of FIG. 8a, in accordance with the general inventive concepts;

FIG. 8c illustrates a third perspective view of the first channel with trap door feature of FIG. 8a, in accordance with the general inventive concepts;

FIG. 9 illustrates another exemplary embodiment of an endoscopic device, in accordance with the general inventive concepts;

FIG. 10 illustrates a further exemplary embodiment of an endoscopic device, in accordance with the general inventive concepts;

FIG. 11 illustrates an exemplary embodiment of a biopsy valve, in accordance with the general inventive concepts;

FIG. 12 illustrates another exemplary embodiment of a biopsy valve, in accordance with the general inventive concepts;

FIG. 13 illustrates an exemplary embodiment of sleeve/cap, in accordance with the general inventive concepts;

FIG. 14 illustrates another exemplary embodiment of sleeve/cap, in accordance with the general inventive concepts;

FIG. 15 illustrates another exemplary embodiment of an endoscopic device with a conforming sleeve, in accordance with the general inventive concepts;

FIG. 16 illustrates a further exemplary embodiment of an endoscopic device, in accordance with the general inventive concepts;

FIG. 17 illustrates a flexible and articulating embodiment of the endoscopic device of FIG. 16, in accordance with the general inventive concepts;

FIG. 18 illustrates an exemplary embodiment of a distal cap, in accordance with the general inventive concepts;

FIG. 19 illustrates a second exemplary embodiment of a distal cap, in accordance with the general inventive concepts;

FIG. 20 illustrates a third exemplary embodiment of a distal cap, in accordance with the general inventive concepts;

FIG. 21 illustrates a fourth exemplary embodiment of a distal cap, in accordance with the general inventive concepts;

FIG. 22 illustrates a fifth exemplary embodiment of a distal cap, in accordance with the general inventive concepts;

FIG. 23 illustrates another exemplary embodiment of an endoscopic device, in accordance with the general inventive concepts;

FIG. 24 illustrates an exemplary embodiment of a balloon, in accordance with the general inventive concepts;

FIG. 25 illustrates an exemplary embodiment of an elevator for an endoscopic device, in accordance with the general inventive concepts;

FIG. 26 illustrates an exemplary embodiment of a means for securing an endoscope at a distal end of an endoscopic device, in accordance with the general inventive concepts;

FIG. 27 illustrates a second exemplary embodiment of a means for securing an endoscope at a distal end of an endoscopic device, in accordance with the general inventive concepts;

FIG. 28 illustrates a third exemplary embodiment of a means for securing an endoscope at a distal end of an endoscopic device, in accordance with the general inventive concepts;

FIG. 29 illustrates a fourth exemplary embodiment of a means for securing an endoscope at a distal end of an endoscopic device, in accordance with the general inventive concepts;

FIG. 30 illustrates a fifth exemplary embodiment of a means for securing an endoscope at a distal end of an endoscopic device, in accordance with the general inventive concepts;

FIG. 31 illustrates a sixth exemplary embodiment of a means for securing an endoscope at a distal end of an endoscopic device, in accordance with the general inventive concepts;

FIG. 32 illustrates a seventh exemplary embodiment of a means for securing an endoscope at a distal end of an endoscopic device, in accordance with the general inventive concepts;

FIG. 33 illustrates an eighth exemplary embodiment of a means for securing an endoscope at a distal end of an endoscopic device, in accordance with the general inventive concepts;

FIG. 34 illustrates a ninth exemplary embodiment of a means for securing an endoscope at a distal end of an endoscopic device, in accordance with the general inventive concepts;

FIG. 35 illustrates a tenth exemplary embodiment of a means for securing an endoscope at a distal end of an endoscopic device, in accordance with the general inventive concepts;

FIG. 36 illustrates an eleventh exemplary embodiment of a means for securing an endoscope at a distal end of an endoscopic device, in accordance with the general inventive concepts;

FIG. 37 illustrates a twelfth exemplary embodiment of a means for securing an endoscope at a distal end of an endoscopic device, in accordance with the general inventive concepts;

FIG. 38a illustrates a further exemplary embodiment of a means for securing an endoscope at a distal end of an endoscopic device, in accordance with the general inventive concepts;

FIG. 38b illustrates a perspective view of the means for securing an endoscope at a distal end of FIG. 38a in a securing position;

FIG. 39a illustrates a side view of an exemplary embodiment of a means for securing an endoscope at a distal end of an endoscopic device, in accordance with the general inventive concepts;

FIG. 39b illustrates a perspective view of the exemplary embodiment of a means for securing an endoscope at a distal end of FIG. 39a;

FIG. 39c illustrates a second perspective view of the exemplary embodiment of a means for securing an endoscope at a distal end of FIG. 39a;

FIG. 40a illustrates a side view of an exemplary embodiment of a means for securing an endoscope at a distal end of an endoscopic device, in accordance with the general inventive concepts;

FIG. 40b illustrates a perspective view of the exemplary embodiment of a means for securing an endoscope at a distal end of FIG. 40a;

FIG. 40c illustrates a second perspective view of the exemplary embodiment of a means for securing an endoscope at a distal end of FIG. 40a;

FIG. 41 illustrates an exemplary embodiment of an endoscopic device attached to a first channel with no additional tethers between the proximal and distal ends where the endoscopic device is attached to the first channel, in accordance with the general inventive concepts;

FIG. 42a illustrates a side view of the endoscopic device attached to the first channel of FIG. 41 with aligned measuring points, in accordance with the general inventive concepts;

FIG. 42b illustrates a second side view of the exemplary embodiment of the endoscopic device attached to the first channel of FIG. 42a, in accordance with the general inventive concepts;

FIG. 42c illustrates a third side view of the exemplary embodiment of the endoscopic device attached to the first channel of FIG. 42a, in accordance with the general inventive concepts;

FIG. 43 illustrates a perspective view of an exemplary embodiment of an endoscopic device attached to an endoscope at its proximal end, in accordance with the general inventive concepts;

FIG. 44 illustrates a second perspective view of the endoscopic device of FIG. 43 attached at its proximal end to the endoscope, in accordance with the general inventive concepts;

FIG. 45 illustrates exemplary embodiments of a connecting part used for securing or otherwise attaching the endoscopic device to an endoscope and/or first channel, in accordance with the general inventive concepts;

FIG. 46 illustrates additional exemplary embodiments of a connecting part with a corresponding strap with one or more openings corresponding to the connecting part size and/or shape, in accordance with the general inventive concepts;

FIG. 47 illustrates exemplary embodiments of a connecting band for use with a securing and/or mounting means for attaching one or more embodiments of the endoscopic device to an endoscope and/or first channel at a distal end and/or proximal end of the endoscopic device, in accordance with the general inventive concepts;

FIG. 48 illustrates exemplary embodiments of an adjusting means for adjusting exemplary embodiments of a band and/or strap for tightening and/or loosening the endoscopic device from the first channel and/or endoscope (via a tightening and/or loosening of the band and/or strap), in accordance with the general inventive concepts;

FIG. 49 illustrates an exemplary embodiment of a means for mounting a proximal end of an endoscopic device to an endoscope or first channel, in accordance with the general inventive concepts;

FIG. 50 illustrates a second exemplary embodiment of a means for mounting a proximal end of an endoscopic device to an endoscope or first channel, in accordance with the general inventive concepts;

FIG. 51 illustrates a third exemplary embodiment of a means for mounting a proximal end of an endoscopic device to an endoscope or first channel, in accordance with the general inventive concepts;

FIG. 52 illustrates a fourth exemplary embodiment of a means for mounting a proximal end of an endoscopic device to an endoscope or first channel, in accordance with the general inventive concepts;

FIG. 53 illustrates a fifth exemplary embodiment of a means for mounting a proximal end of an endoscopic device to an endoscope or first channel, in accordance with the general inventive concepts;

FIG. 54 illustrates a sixth exemplary embodiment of a means for mounting a proximal end of an endoscopic device to an endoscope or first channel, in accordance with the general inventive concepts;

FIG. 55 illustrates a seventh exemplary embodiment of a means for mounting a proximal end of an endoscopic device to an endoscope or first channel, in accordance with the general inventive concepts;

FIG. 56 illustrates an eight exemplary embodiment of a means for mounting a proximal end of an endoscopic device to an endoscope or first channel, in accordance with the general inventive concepts;

FIG. 57 illustrates a ninth exemplary embodiment of a means for mounting a proximal end of an endoscopic device to an endoscope or first channel, in accordance with the general inventive concepts;

FIG. 58 illustrates a tenth exemplary embodiment of a means for mounting a proximal end of an endoscopic device to an endoscope or first channel, in accordance with the general inventive concepts;

FIG. 59 illustrates an eleventh exemplary embodiment of a means for mounting a proximal end of an endoscopic device to an endoscope or first channel, in accordance with the general inventive concepts;

FIG. 60 illustrates a twelfth exemplary embodiment of a means for mounting a proximal end of an endoscopic device to an endoscope or first channel, in accordance with the general inventive concepts;

FIG. 61 illustrates a thirteenth exemplary embodiment of a means for mounting a proximal end of an endoscopic device to an endoscope or first channel, in accordance with the general inventive concepts;

FIG. 62 illustrates a fourteenth exemplary embodiment of a means for mounting a proximal end of an endoscopic device to an endoscope or first channel, in accordance with the general inventive concepts;

FIG. 63 illustrates a fifteenth exemplary embodiment of a means for mounting a proximal end of an endoscopic device to an endoscope or first channel, in accordance with the general inventive concepts;

FIG. 64 illustrates a sixteenth exemplary embodiment of a means for mounting a proximal end of an endoscopic device to an endoscope or first channel, in accordance with the general inventive concepts;

FIG. 65 illustrates a seventeenth exemplary embodiment of a means for mounting a proximal end of an endoscopic device to an endoscope or first channel, in accordance with the general inventive concepts;

FIG. 66 illustrates an eighteenth exemplary embodiment of a means for mounting a proximal end of an endoscopic device to an endoscope or first channel, in accordance with the general inventive concepts;

FIG. 67 illustrates a nineteenth exemplary embodiment of a means for mounting a proximal end of an endoscopic device to an endoscope or first channel, in accordance with the general inventive concepts;

FIG. 68 illustrates another exemplary embodiment of an endoscopic device in operation, in accordance with the general inventive concepts; and

FIG. 69 illustrates a further exemplary embodiment of an endoscopic device in operation, in accordance with the general inventive concepts.

### DETAILED DESCRIPTION

The general inventive concepts will be understood more fully from the detailed description given below and from the accompanying drawings of the various aspects and implementations of the disclosure. This should not be taken to limit the general inventive concepts to the specific aspects or implementations, which are being provided for explanation and understanding only.

In discussing the exemplary embodiments herein, the terms “proximal” and “distal” may often be used. These terms are used to describe a position or a direction with reference to the operator of the instrument. For example, the proximal position or proximal direction is toward the user or operator of the instrument, and the distal position or direction is away from the user or operator of the instrument, i.e., position or direction toward the object which the operator is attempting to grasp, retain, and/or view.

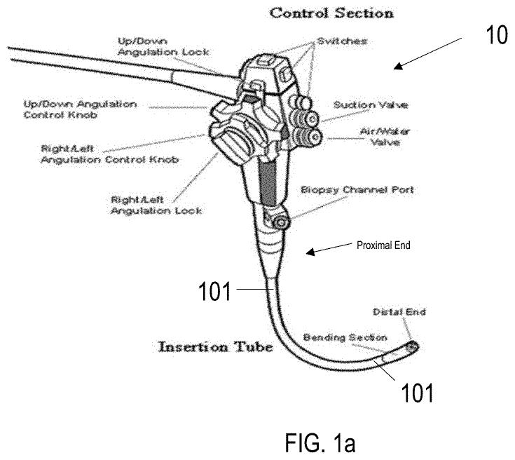

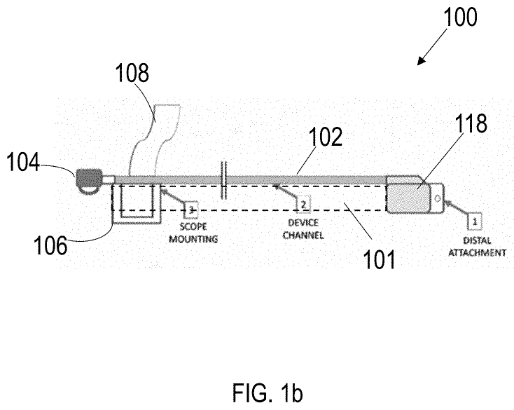

Referring now to the drawings, which are for purposes of illustrating exemplary embodiments of the subject matter herein only, and not for limiting the same, FIG. 1a shows an exemplary embodiment of an endoscope 10, and FIG. 1b shows an exemplary embodiment of an endoscopic device 100 for use with one or more endoscopic instruments (including the endoscope 10 and other devices such as a snare, injection needle, hemostasis clip etc.). Endoscopic instruments are traditionally fed through a default channel 101 of the endoscope (also referred to as a first channel), which may be the primary channel. In some embodiments, the endoscope 10 may be a single endoscope configuration or a dual channel endoscope configuration.

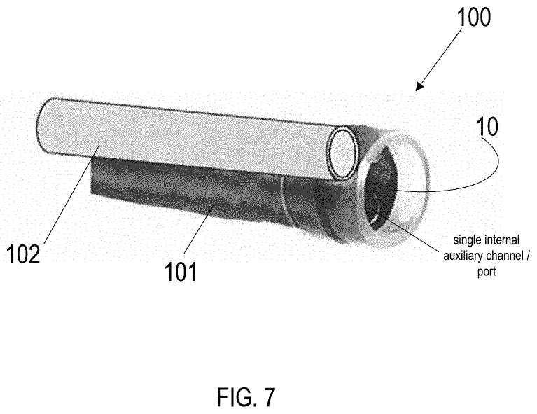

As illustrated in FIG. 1b, the endoscopic device 100 may include at least one additional channel (also referred to as a catheter 102) which may be secured via a securing means to the endoscope 10. In some embodiments, a plurality of additional channels (i.e., two or more the catheters 102) may be provided. It should be appreciated that endoscopic devices with an additional channel (i.e., the catheter 102) differ from dual channel endoscope instruments in the art in that the catheter 102 may be externally attached to the endoscope 10 and/or the first channel 101, while a dual channel endoscope is merely an endoscope have one more internal auxiliary channel/port than traditionally found in single channel endoscopes (e.g., endoscope 10 as shown in FIG. 7). The additional channel endoscopic device 100 (with catheter 102) may be removably attached to an endoscope to provide a separate lumen for instrument access and/or suction. The benefit of the catheter 102 is that it can be used in conjunction with an endoscope 10 to provide an additional channel for instrument access and/or suction.

In some embodiments, a diameter of the catheter 102 may be less than a diameter of the first channel 101. In some embodiments, the catheter 102 may be more flexible than the first channel 101. Additionally, or alternatively, the endoscope pathways in a dual channel endoscope instrument may be contiguously attached along a length of the dual channel endoscope instrument within the first channel 101. In some embodiments, the catheter 102 may not be attached to the endoscope 10 and/or first channel 101 between the proximal and distal end connections.

It should be appreciated that the catheter 102 may be provided to allow users (e.g., physicians) to seamlessly and/or simultaneously use additional endoscopic instruments via the catheter 102 during an endoscopic procedure, and without having to remove, for example, the endoscope 10 used in the first channel 101. For example, if during a endoscopic procedure, blood or debris inhibits the user's view, the user can now utilize a system (e.g., a suction system 200) via the catheter 102 to suction away the blood or debris.

Examples of one or more endoscopic instruments (also referred to as accessories) that may be used via the catheter 102 may include biopsy forceps, Lariat® cutting snares, Raptor® Grasping Tools, and Carr-Locke® Injection Needles. However, it should be appreciated that other devices known in the art may be used via the catheter 102.

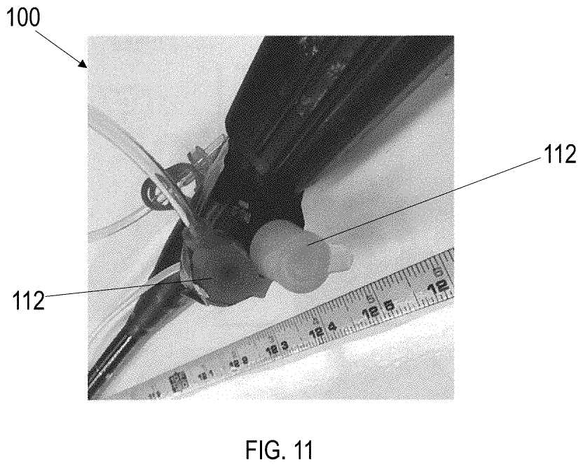

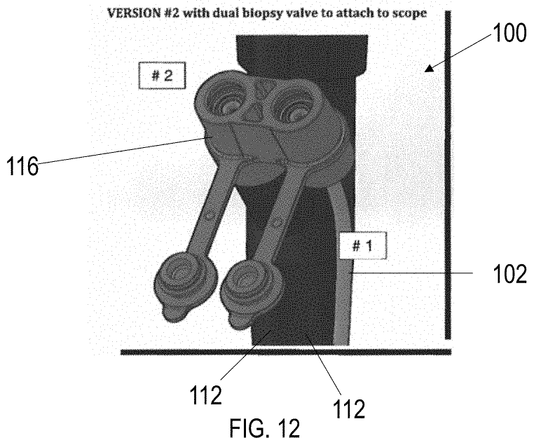

In some embodiments, the endoscopic device 100 may include one or more biopsy valves 104 secured or otherwise attached to the catheter 102 at an end opposite the distal end (e.g., a proximal end). In some embodiments, at least one of the biopsy valves 104 may be a dual biopsy valve 116 (FIGS. 11-12). A dual biopsy valve 116 attachment may be provided to close a dormant channel via an integral lid, which may assist to limit fluid leakage, or to open the dormant channel if a larger orifice is desired to increase efficiency.

In some exemplary embodiments, the biopsy valve 104 (e.g., the dual biopsy valve 116) may be provided as a means for securing or otherwise attaching the catheter 102 to the first channel 101. It should be appreciated that the design of the dual biopsy valve 116 may assist to stabilize endoscopic instruments due to its arrangement and frictional features.

In some embodiments, the dual biopsy valve 116 may be arranged such that it limits or otherwise restricts movement of the tube, and thereby allows for rotational stabilization. For example, if an accessory is rotatable, the position of the dual biopsy valve stabilizes the accessory by limiting its rotational movement. Additionally, or alternatively, the position of the biopsy valve may limit or otherwise restrict a proximal movement of the tube.

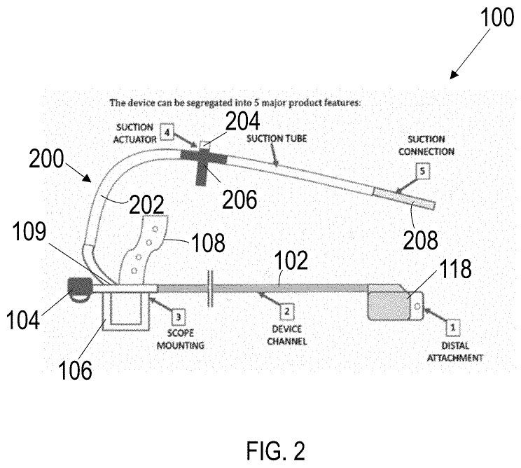

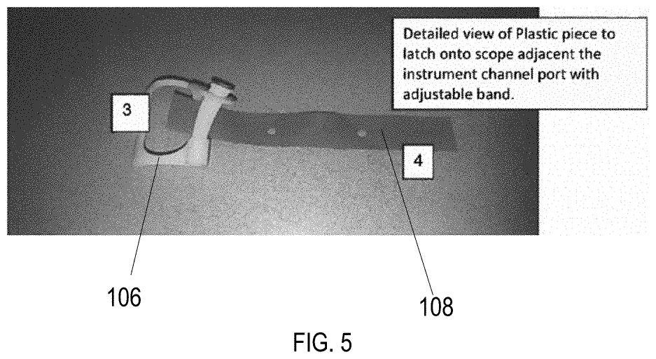

With continue reference to FIGS. 1a-b, and now with reference to FIGS. 2-6, the endoscopic device 100 may include a means for securing the endoscope 10 (and/or first channel 101) to the endoscopic device 100. In some embodiments, the means for securing the endoscope 10 to the endoscopic device 100 may include a mounting brace 106 sized and/or shaped for attaching or otherwise contacting the endoscope 10 and a strap or band 108 sized and/or shaped for securing or otherwise attaching the mounting brace 106 to the endoscope 10. Additionally, or alternatively, the biopsy valve (e.g., the dual biopsy valve) may be provided for securing the endoscope 10 to the endoscopic device 100.

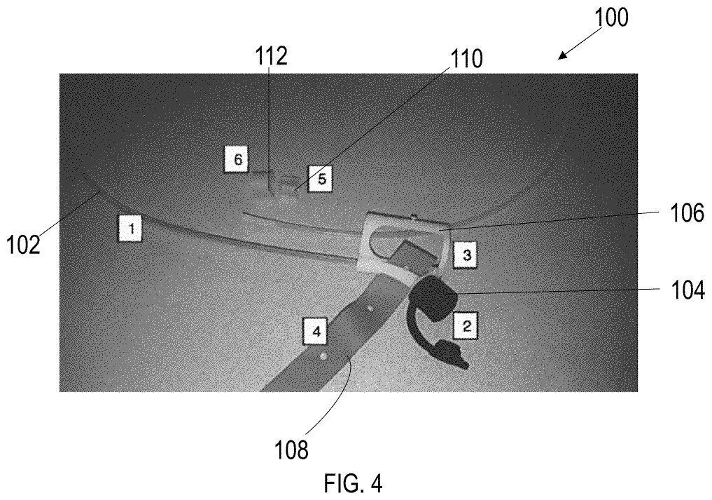

It should be appreciated that in some embodiments, for example, the endoscope 10 (or more particularly the endoscope 10 head) may be covered by a sleeve (e.g., a protective sleeve 110 (FIG. 4) or similar covering) prior to attaching the endoscope 10 to the endoscopic device 100.

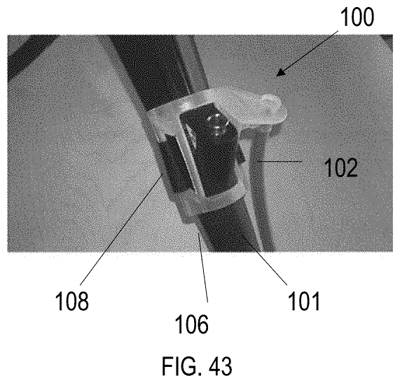

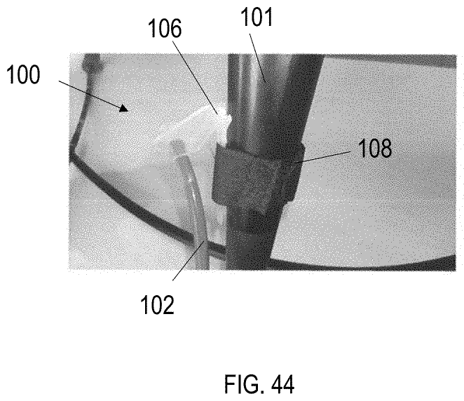

The mounting brace 106 may be formed of a plastic or similar polymer material, although other materials may be used for forming the mounting brace 106. The mounting brace 106 may latch onto the endoscope 10, and the band 108 may be used to secure the endoscope 10 adjacent to the catheter 102 (e.g., FIG. 43 and FIG. 44 show at least one embodiment of the mounting brace 106 and band 108 securing the first channel 101 to the endoscopic device 100).

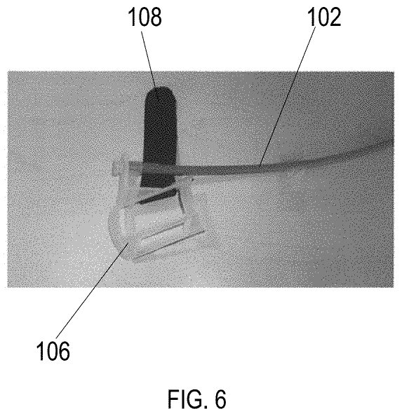

In some embodiments, the band 108 may be adjustable to accommodate different endoscope 10 sizes and may include one or more holes for adjusting the band around the endoscope 10 and the catheter 102. In some embodiments, as shown in FIG. 6, the band 108 or at least a portion of the band 108 may be a hook and loop fastener.

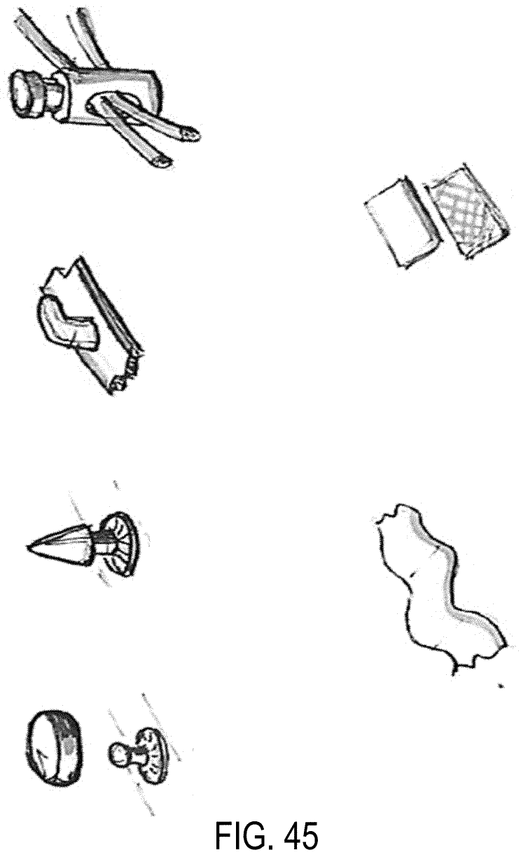

With continued reference to the figures, and with reference now to FIG. 45, exemplary embodiments of a connecting part for securing the endoscopic device 100 to the endoscope 10 (e.g., at the head and/or handle of the endoscope 10) and/or the first channel 101 is provided. In the exemplary embodiment of FIG. 45, one or more of the connecting parts may be a snap (which may include two or more mating pieces (two shown)), a stud, and/or a hook. Additionally, or alternatively, the connecting part may be formed of a self-sticking material and/or may include a hook and loop fastener. It should be appreciated that other types of connecting parts (not shown) but known in the art may be used with a securing means for attaching the endoscopic device 100 to the first channel 101 and/or endoscope 10.

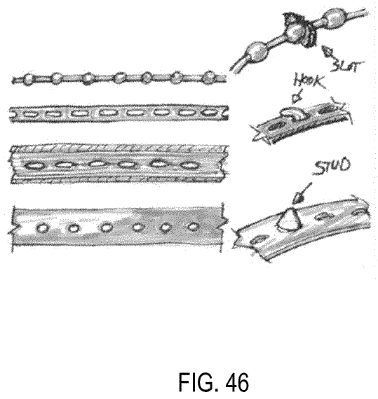

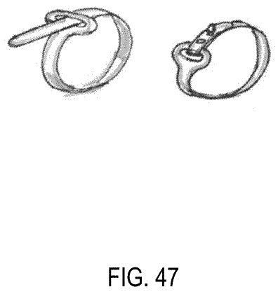

With reference now to FIG. 46, a further embodiment of a connecting part is shown with a corresponding band 108 and/or strap. It should be appreciated that the band 108 and/or strap used with the connecting parts may include one or more openings (FIG. 47) for securing the multiple bands 108 and/or straps together (e.g., via the connecting parts). It should be appreciated that the size and shape of one or more of the openings may corresponding to the connecting part size and/or shape.

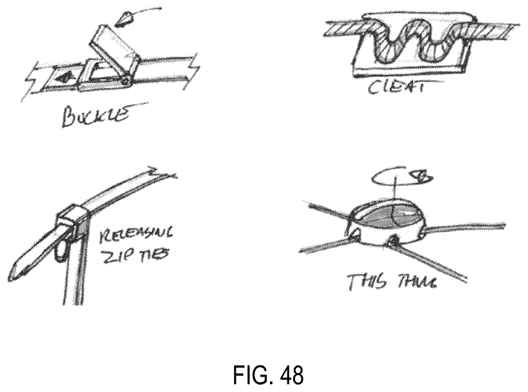

With reference now to FIG. 48, a means for adjusting (adjusting means) one or more bands 108 is provided. In the embodiments shown in FIG. 48, the adjusting means may be a buckle, a cleat, a zip tie (e.g., a releasable zip-tie), and/or a rotating clasp configurable to reduce and/or increase a length of the band 108 by rotating the clasp in either direction. It should be appreciated that the adjusting means may be used with the securing means at the distal end of the endoscopic device 100 and/or the mounting means at the proximal end of the endoscopic device 100.

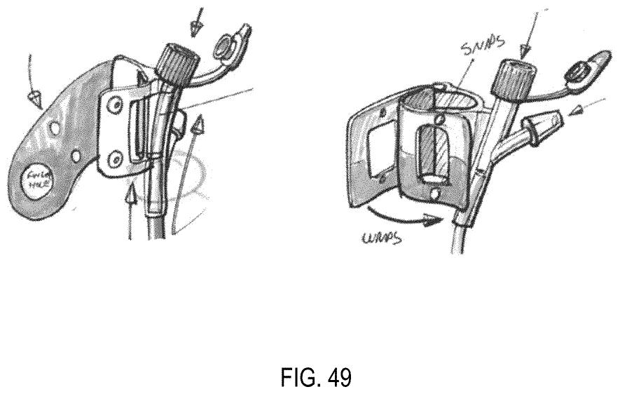

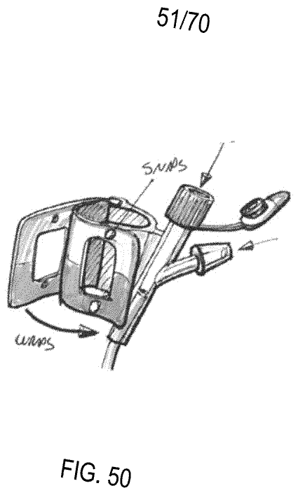

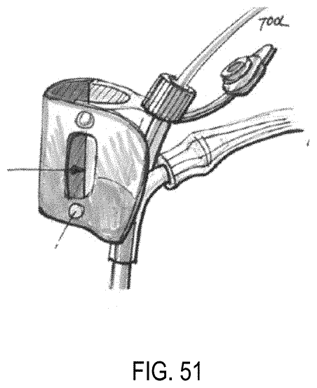

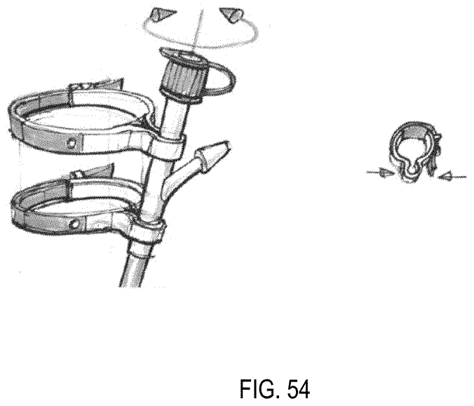

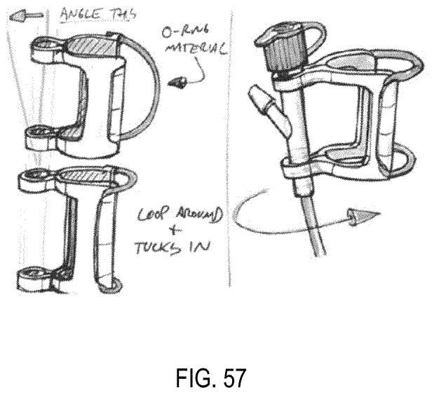

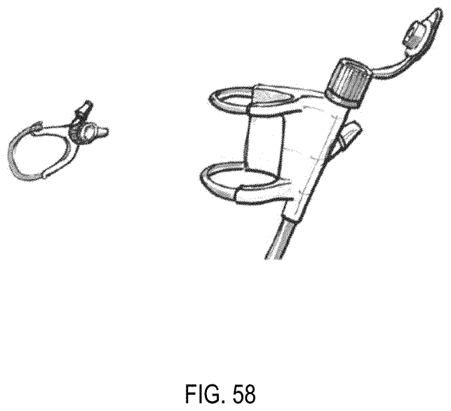

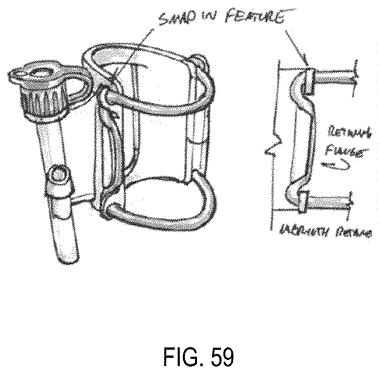

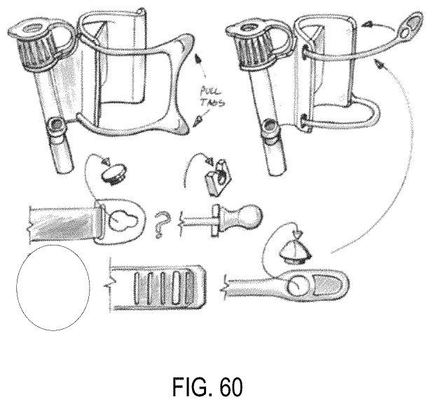

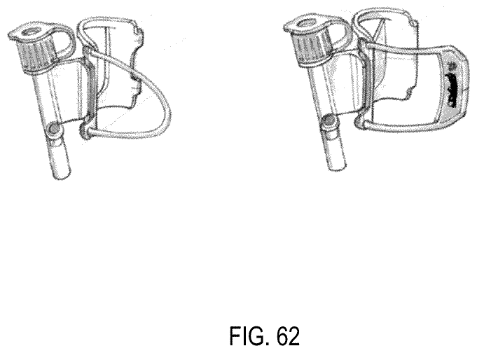

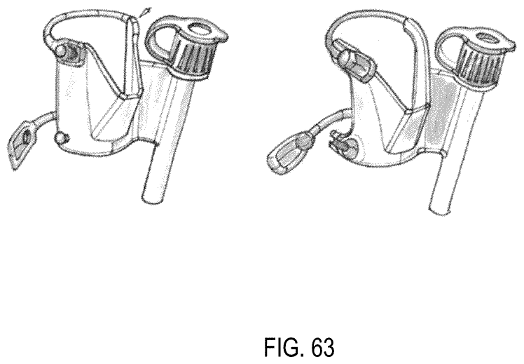

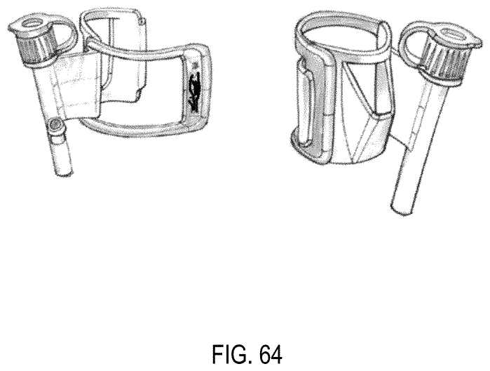

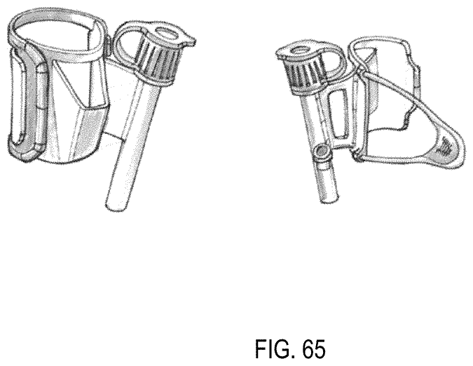

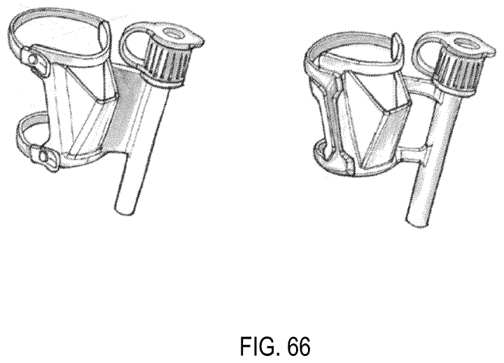

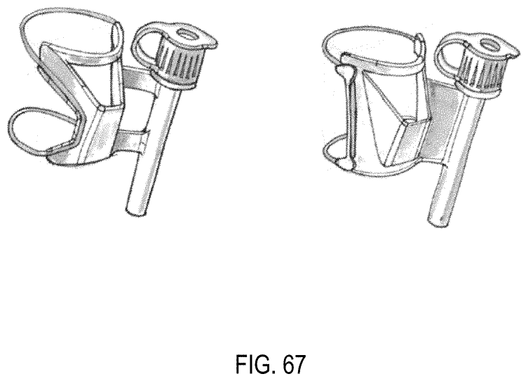

With continued reference to the figures, and with reference now to FIGS. 49-67, exemplary embodiments of a means for mounting (mounting means) the proximal end of the endoscopic device 100 to the first channel 101 and/or endoscope 10 handle (not shown) are provided. In some embodiments, the mounting means may be a mounting bracket having a first portion for connecting to a proximal end of the catheter 102, and a second portion for connecting to the first channel 101 and/or endoscope 10.

In some embodiments, the mounting bracket may include a brace and band 108 and/or strap. The brace may be rigid or flexible, and be shaped or otherwise sized to interface with the first channel 101 and/or the catheter 102.

Additionally, or alternatively, the mounting bracket may include or otherwise incorporate one or more connecting parts (FIG. 45) with the bands 108 and/or strap.

In some embodiment, for example as shown in FIGS. 49-67, the mounting bracket may include a combination of connecting parts (e.g., a stud), a band 108 or strap, and mounting brace for securing the mounting bracket to the first channel 101. In these exemplary embodiments, the connecting parts may be formed on the mounting brace with one or more corresponding openings formed in the band 108 for receiving the connecting parts therebetween.

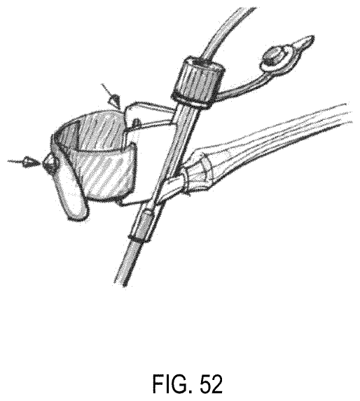

In the embodiment of FIG. 52, the connecting part is formed at one end of the band 108, while the opposite end of the band 108 includes an opening or similar void for at least partially receiving the connecting part therethrough for securing the band 108. In this embodiment, opposite sides of the mounting brace may include an opening for receiving the band 108 therethrough. In some embodiments, for example, two bands 108 may be secured to opposite sides of the mounting brace in lieu of the band openings in opposite sides of the mounting brace. In this embodiment, the connecting part may be at one end of one of the bands 108, while a corresponding opening is position at an end of the other band 108.

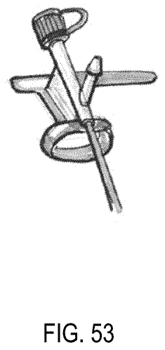

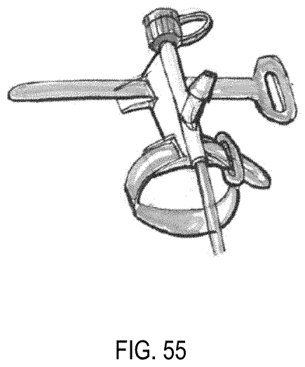

In some embodiment, two or more bands 108 at opposite ends of a mounting brace may be used for securing the endoscopic device 100 to the first channel 101 and/or endoscope 10. For example, the embodiment of FIG. 53 illustrates two bands 108 at opposite ends of a narrow mounting brace. In this embodiment, a first band 108 may be formed on an outer part of the mounting brace, while a second band may be formed beneath the mounting brace (e.g., on another part of the mounting bracket). In some embodiment, the narrower mounting brace may include one or more openings (two shown in FIG. 55) for receiving one or more straps therebetween.

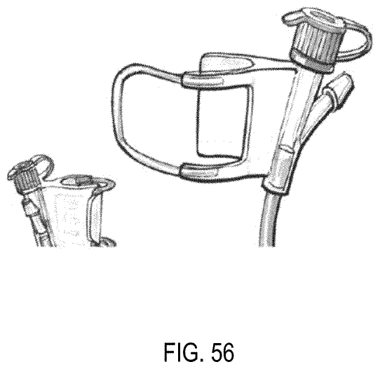

In some exemplary embodiments, for example, as shown in FIGS. 56-59, and FIG. 62 the mounting brace may be configured for securing one or more bands 108 and/or straps thereto in lieu of or in addition to using one or more connecting parts. For example, the mounting brace may include an outer flange (e.g., a retaining flange) or hook-shaped portion for attaching one or more bands thereto and for securing the endoscopic device 100 to the endoscope 10 and/or first channel 101.

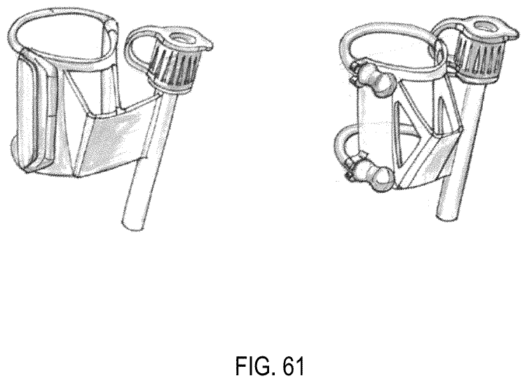

In some embodiments, the mounting bracket may include one or more openings adapted or otherwise shaped to conform to one or more parts of the endoscope 10 (e.g., a handle (not shown)) at a proximal end of the endoscope. In some embodiments, the opening may be formed within a conforming structure. In some embodiments, for example, as shown in FIG. 61, the conforming structure may include one or more openings on opposites sides of the conforming structure (two shown in FIG. 61). Additionally, or alternatively, the conforming structure may be part of the mounting brace. In this embodiment, the mounting brace may be shaped or otherwise sized to conform to the endoscope and/or a handle of the endoscope at the proximal end.

It should be appreciated that any one or more of the connecting portions and/or bands 108 may be used as a securing means (or part thereof) at the distal end of the endoscopic device and/or with any mounting means (or part thereof) at the proximal end of the endoscopic device. Additionally, or alternatively, any combination of connecting portions and/or bands 108 may be used for the securing means and/or mounting means.

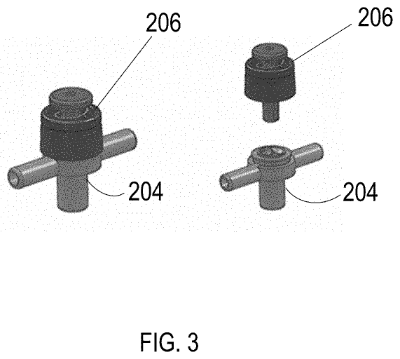

With continued reference to the figures, one or more endoscopic systems may be operably connected to the endoscopic device 100, for example, via the catheter 102. For example, as illustrated in the exemplary embodiments of FIGS. 2 and 3, at least one of the endoscopic systems may be a suction system 200. The suction system 200 may be operably connected to the endoscopic device 100 via the catheter 102. In the embodiment of FIG. 2, the suction system 200 may include one or more suction tubes 202 operably connected to a suction unit (not shown) via a suction actuator 204, a suction valve 206, and a suction connecting part 208.

Additionally, or alternatively, a y-port 109 (FIG. 2) may be provided for connecting the suction system 200 to the endoscopic device 100. It should be appreciated that suctioning during an endoscopic procedure (e.g., an endoscopic submucosal dissection (ESD) procedure) may be controlled via the suction valve 206.

With reference now to FIG. 7, in some exemplary embodiments, a distal opening of the first channel 101 (e.g., with endoscope 10) may be larger than a distal opening of the catheter 102. It should be appreciated that, in some embodiments, the opening for both the first channel 101 and the catheter 102 may be similar, or dissimilar, depending on how the endoscopic device 100 will be used. Additionally, or alternatively, the distal opening of catheter 102 may be larger than the distal opening of the first channel 101.

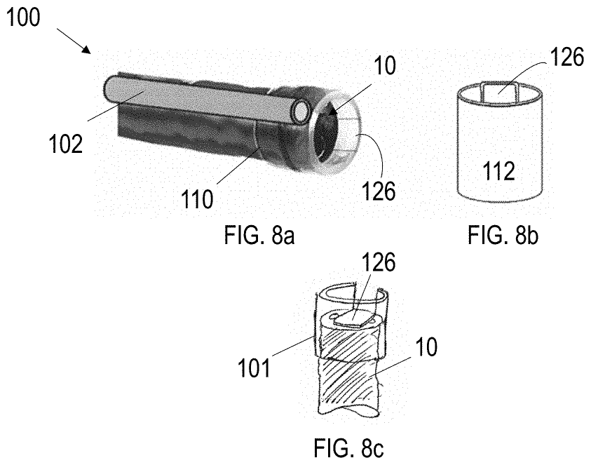

With reference now to FIGS. 8a-8c, in some embodiments, a distal end of the first channel 101 and/or the catheter 102 may include a trap door feature. The trap door feature may be provided to assist with keeping the catheter 102 clear of debris and dry. The trap door feature is advantageous for procedures with excessive moisture. For achieving the trap feature, a notched end 126 flappable into an open and closed position may be provided at the distal end of the first channel 101 (FIG. 8a) or similar outer sleeve (FIG. 8c), and/or the catheter 102 (not shown), and/or in one or more caps at the distal end (e.g., a distal cap 112) (FIG. 8b). It should be appreciated that the trap door feature may also allow for the cap to flex outwardly. In some embodiments, a pull wire (not shown) may be provided to generate the outwardly flex motion, and in some embodiments, for opening and closing the notched end 126 (trap door). The pull wire may travel to the proximal end of the endoscopic device 100.

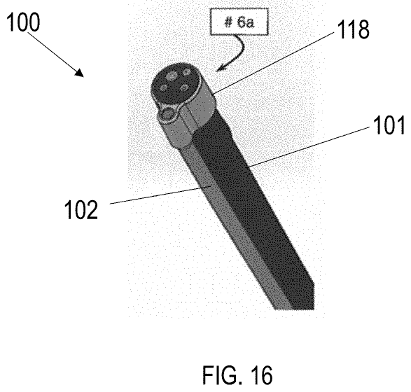

With continued reference to the figures, in some embodiments, the means for securing one or more catheters 102 to the first channel 101 and/or endoscope 10 may be an attachment cap or sleeve (e.g., the distal cap 112 (FIG. 4) and/or an attachment sleeve 118 (FIG. 16). In some embodiments, the distal cap 112 and/or the catheter 102 may include an elastomer flap (not shown). The elastomer flap may be provided to partially or fully block (close) an opening at the distal end of the catheter 102. In some embodiments, a pull wire may be attached to the elastomer flap for opening the flap as needed.

One or more of the attachment sleeve 118 and/or distal cap 112 may be molded into a desired shape (e.g., a “c”-shape and/or at least a partially cylindrical shape) for fitment around the distal end of the endoscopic device 100 (e.g., around the catheter 102 and/or the first channel 101). It should be appreciated that the attachment sleeve 118 and/or distal cap 112 may also be formed by other means known in the art for forming sleeves and/or caps (e.g., 3D printing).

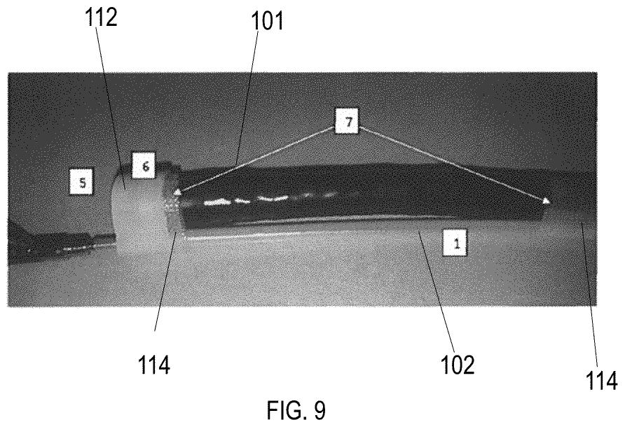

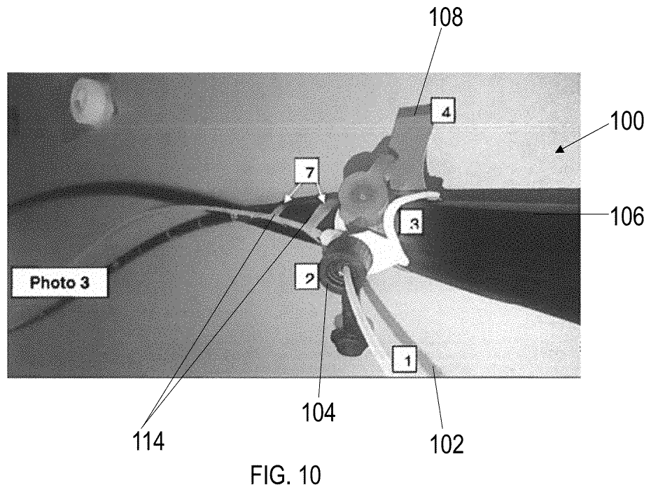

With reference now to FIG. 9, in some embodiment, a tape 114 may be used to secure or otherwise attach the catheter 102 to the first channel 101 and/or endoscope 10 at various points along a length of the endoscopic device 100. For example, the embodiment of FIGS. 9-10 illustrate at least two pieces of tape 114 attaching the catheter 102 to the endoscope 10. FIG. 9 shows the tape 114 attaching the catheter 102 to the first channel 101 of the endoscope 10 at the distal end and at a second point between the distal and proximal end of the endoscope 10. In the embodiment shown, the second point is nearest to the distal end than the proximal end of the endoscope 10. In the embodiment of FIG. 10 at least two pieces of tape 114 are shown attaching the first channel 101 to the catheter 102 nearest to the proximal end of the endoscopic device 100 and endoscope 10.

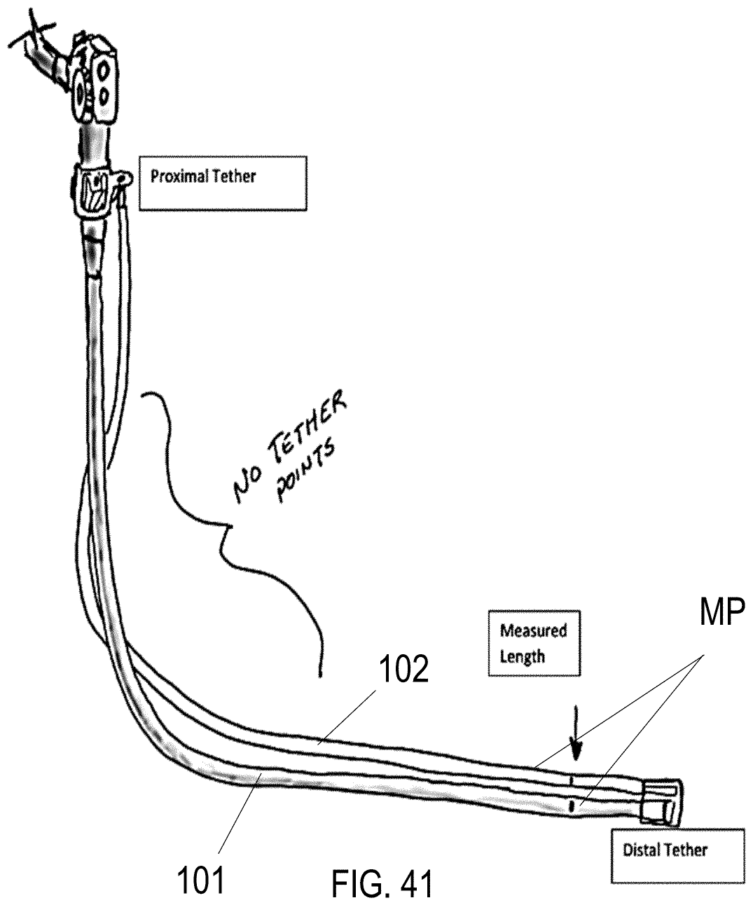

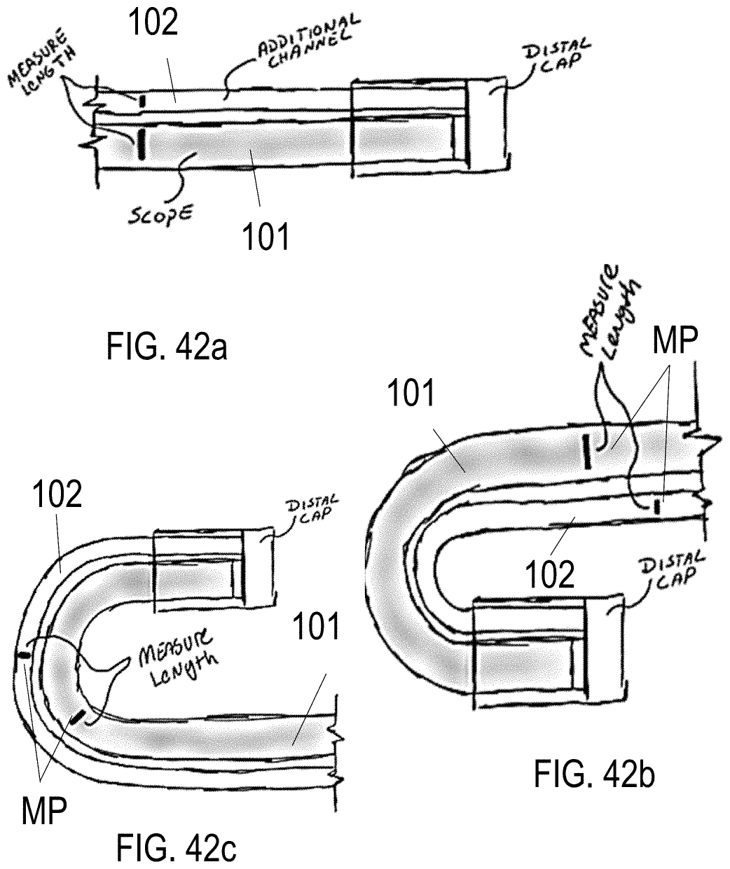

It should be appreciated that in some embodiments, for example, as shown in the exemplary embodiments of FIGS. 41 and 42a-c, no additional tether points may be provided between the connection points (tethers) at the distal and proximal ends. By providing embodiments with no additional tethers, the length of the catheter 102 may be increased (i.e., the catheter 102 may have an extra length), which allows for easier maneuvering of the catheter 102.

Additionally, with no additional tether points, the catheter 102 is able to move at a different rate than the endoscope 10 (and/or first channel 101) in torturous retroflex configurations. Because the catheter 102 may be orientated in any clocking position on the distal end of the first channel 101, the bend radius of the catheter 102 can either be greater or less than the actual bend radius of the first channel 101 should the catheter 102 lay on the inner (FIG. 42b) or outer (FIG. 42c) parameters of the first channel 101.

It should be appreciated that, as illustrated in FIGS. 42b-c, the equal measured length (as indicated by MP in FIG. 42a-c) on the catheter 102 changes as the travel distance around the bend changes. It should be further be appreciated that, by not including additional tether points, the catheter 102 can freely travel at different lengths without notice from the endoscope user. It should further be appreciated that the catheter 102 is now allowed to freely find its natural path of least resistance and may shift when the endoscope 10 is either in retroflexion or in a torturous configuration through the bodily cavity (not shown). Less tether points equals to less points of stress or restriction thus reducing or eliminating the potential for kinking and allows for device freedom (insertion and retraction of devices).

In yet a further exemplary embodiment, one or more of the first channel 101 and/or catheter 102 may include one or more openings along a length of the first channel 101 and/or catheter 102 to allow for a more flexible maneuvering of one or more devices extending therethrough.

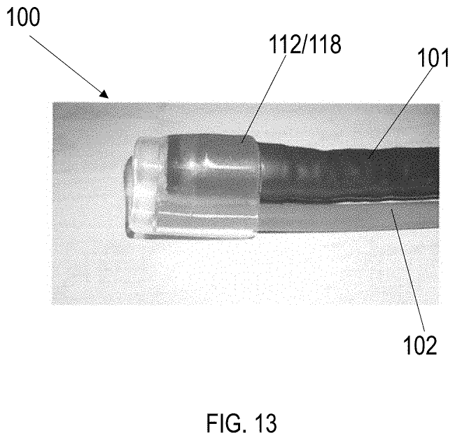

With continued reference to the figures, and now with reference to FIG. 13, the distal cap 112 may include a plurality of openings for attaching the distal cap 112 to distal ends of the endoscope 10 and/or catheter 102.

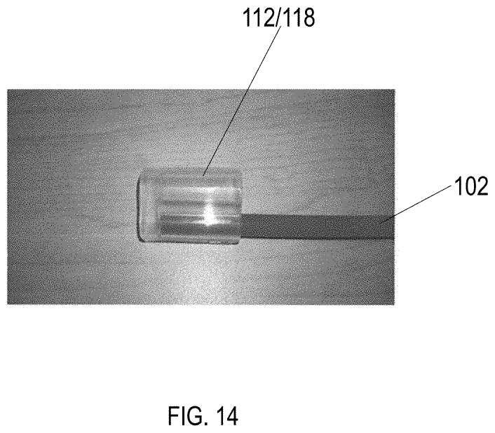

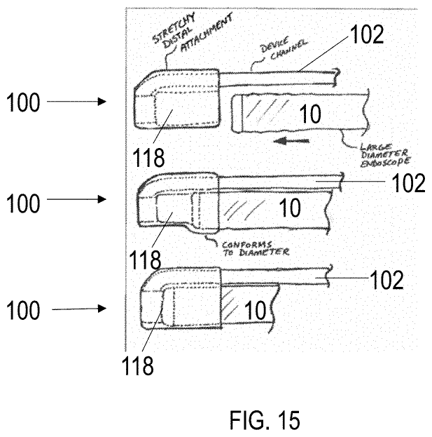

As shown in FIG. 13, at least two of the openings in the distal cap 112 are differently sized to accommodate channels (tubes) of different sizes. For example, in this embodiment, the distal cap 112 includes a larger opening for receiving (or at least partially receiving) parts of an endoscopic instrument (e.g., the first channel 101/endoscope 10) and a smaller opening for receiving (or at least partially receiving) parts of the endoscopic device 100 (e.g., the catheter 102). It should be appreciated that the openings for receiving the first channel 101 and catheter 102 may be the same or a similar size (FIG. 14) based on needs and/or the type of endoscopic procedure being performed. It should be further appreciated that the opening for receiving the catheter 102 may be parallel to a larger opening with an angle of zero to the horizontal axis of the endoscope 10 (FIG. 14) or at a non-zero angle (FIG. 15).

For example, in one exemplary embodiment, an accessory or similar instrument may come out straight from the catheter 102 in the same manner as an endoscopic device would exit the first channel 101.

It should be appreciated that a nonzero angle allows for triangularization of the accessory devices/instruments. A first device comes parallel (to the endoscope) via the endoscope native channel (i.e., the first channel 101) and a second instrument meets or triangulates the first at a designed distance (via the catheter 102), set by the nonzero angle of the opening.

It should be appreciated that the distal cap 112 may be flexibly expandable to conform to a larger diameter first channel 101 and/or catheters 102 for frictionally fitting the first channel 101 and/or catheters 102.

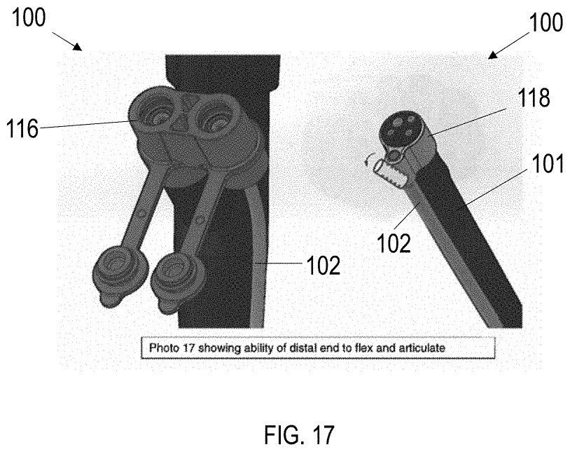

It should be further appreciated that, in addition to flexibility, an elastomer sleeve 118 and/or distal cap 112 allows for articulating a distal end of the endoscopic device 100 (FIG. 17). Providing such flexibility is advantageous because it results in less trauma during insertion of the endoscopic instrument (device) and while the device travels to the target site. The articulation may help to provide leverage as the device in the catheter 102 may now be moveable in a different axis than that of the endoscope 10 itself.

The articulation may also allow for triangulation of the two or more devices exiting from both the first channel 101 and the catheter 102 (or multiple catheters if desired). In this example, at some distance past the distal end of the endoscopic device 100, the 2 or more devices would converge, and may potentially assisting with procedures like ESD. Additionally, an articulating distal end provided by the catheter 102 would provide a user with the ability to maneuver devices independently of the endoscope, thus affording the user more control during the procedure, for example, by providing for an additional axis of movement for the device. In some embodiments, the articulation may be passive and/or active.

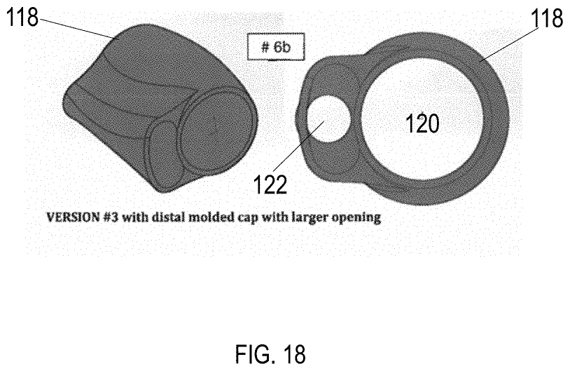

With continued reference to the figures, and now with reference to FIGS. 18-22, one or more openings of the attachment sleeve 118 and/or distal cap 112 may be comprised of different shapes and or sizes for at least partially receiving the first channel 101 (endoscope 10) and/or catheter 102. As shown in FIG. 18, the attachment sleeve 118 may include a first opening 120 for the endoscope 10 (first channel 101) that may be larger than a second opening 122 for the catheter 102. It should be appreciated that different size openings may be provided as needed for using differently sized devices.

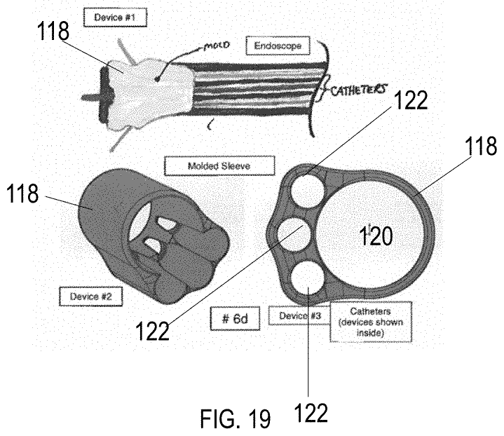

In some embodiments, the attachment sleeve 118 (or distal cap 112) may include a plurality of openings. For example, the embodiment of FIG. 19 shows an attachment sleeve 118 with at least three openings 122 sized and/or shaped for at least partially receiving at least three catheters 102 therebetween. It should be appreciated that the openings 122 may be similarly or dissimilarly sized to each other and/or the opening 120 for the endoscope 10. FIG. 19 shows three opening 122 being similarly sized, with each opening 122 smaller than the opening 120 for the endoscope 10.

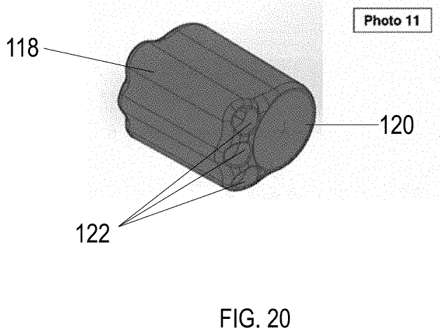

In some embodiments, one or more of the openings of the attachment sleeve 118 may include a cover or flap (e.g., an elastomer flap (FIG. 20)) for at least partially closing the openings, for example, when the endoscopic device 100 and/or specific catheters 102 are not in use.

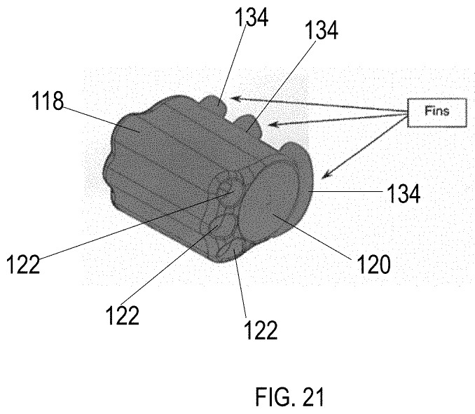

In some exemplary embodiments, as shown in FIG. 21, the attachment sleeve 118 (or distal cap 112) may include one or more fins 134 extending from an exterior (outer) surface of the attachment sleeve 118. Fins may be provided to help move folds that naturally occur in tissues and which may obscure a user's ability to visualize, for example, polyps and/or other cell architecture of interest.

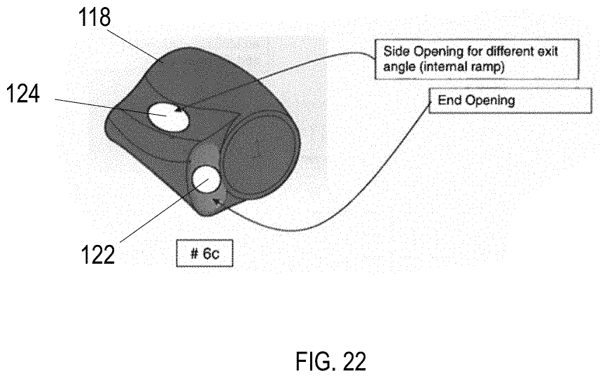

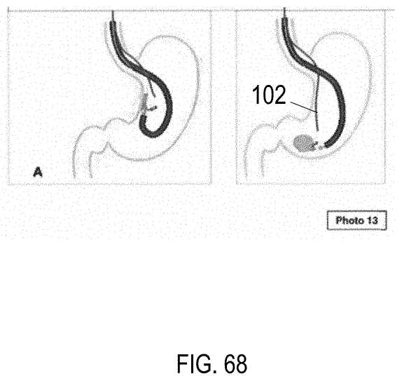

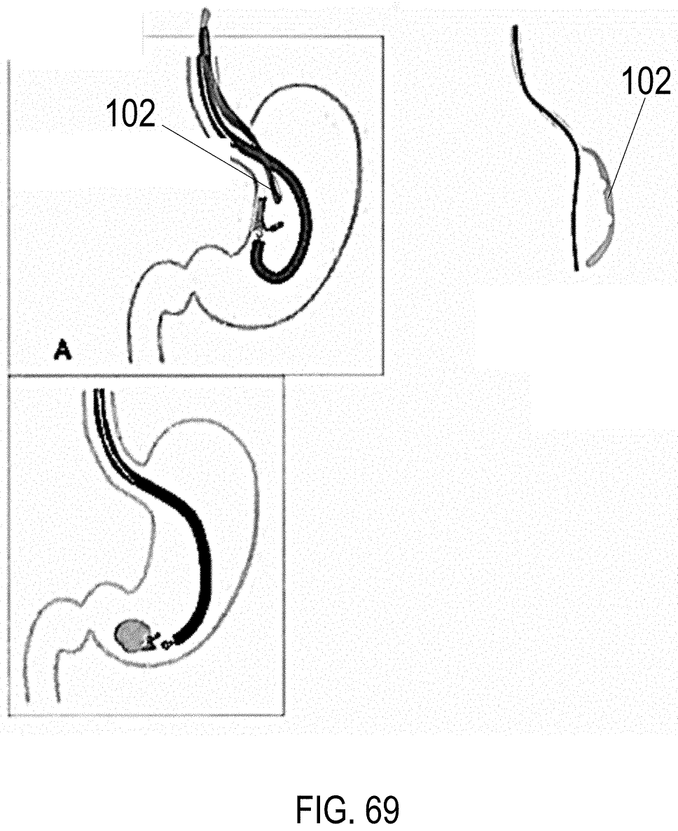

Additionally, or alternatively, the attachment sleeve 118 (or distal cap 112) may include one or more side openings 124 (FIG. 22). The side opening 124 may be provided as an exit opening to allow for one or more different exit angles. This and other exemplary embodiments of the general inventive concepts may be advantageous for procedures where, for example, an anchor is needed to lift or hold the anatomy so a user may perform certain tasks with an additional device using the catheter 102 (e.g., as shown in FIG. 68 and FIG. 69). It should be appreciated that offering different exit angles may also be advantageous because it allows for devices exiting the catheter 102 to be persuaded into other areas (e.g., a camera's field of view) instead of remaining horizontal (i.e., parallel to the endoscope 10 axis).

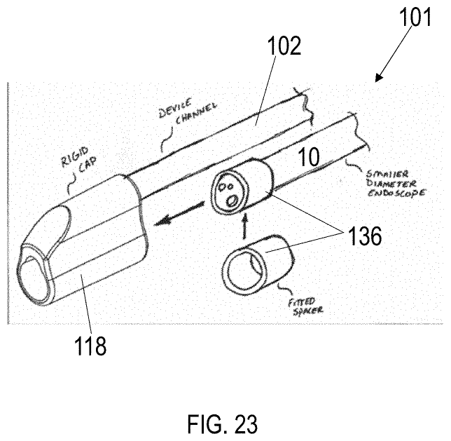

With reference now to FIG. 23, the endoscopic device 100 may include a spacer 136 for fitting one or more of the endoscope 10, first channel 101, and/or catheter 102 into the attachment sleeve 118. The spacer 136 may be comprised of one or more silicones, thermoplastic elastomers (TPE), Flexible PVC, polyurethane, or similar materials.

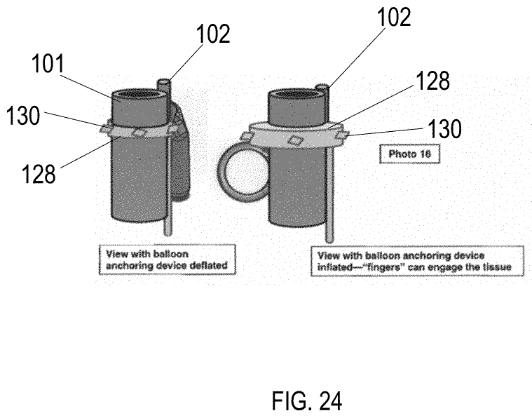

With reference now to FIG. 24, the endoscopic device 100 may include a balloon 128. The balloon 128 may be arranged at or near the distal end of the endoscopic device 100. In some exemplary embodiments, the balloon 128 may be arranged on an external diameter for compressing (upon inflation) devices and/or materials that interface with the endoscope 10.

Additionally, or alternatively, the balloon 128 may include one or more fingers 130 extending outwardly therefrom for engaging tissue in operation. The balloon 128 may be comprised of one or more of a synthetic polyisoprene, TPE, polyurethane, or similar material known in the art for forming balloons.

In some embodiments, one or more balloons may be arranged within the catheter 102 and/or a similar channel (e.g., on an internal diameter of the catheter 102, sleeve, or other channel) for manipulating and/or compressing (upon inflation) devices (e.g., within the catheter 102) and/or materials that interface with the endoscope 10.

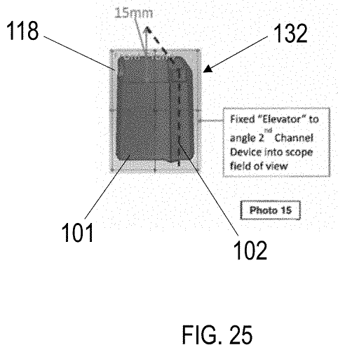

With reference now to FIG. 25, the endoscopic device 100 may include an elevator 132. In some embodiments, the elevator 132 may be provided for angling the catheter 102 into the endoscope 10 field of view and/or for allowing the user to generate device angles different from that of the first channel 101, which may be fixed with the axis of the endoscope 10.

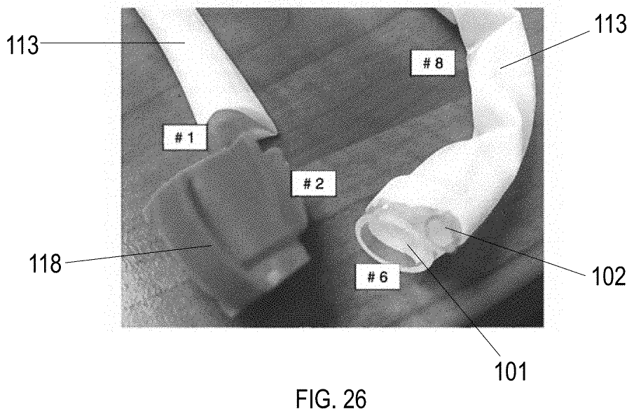

With reference now to FIGS. 26-40, securing or otherwise attaching a distal end of the catheter 102 to the endoscope 10 and/or the first channel 101 may be achieved by additional securing means.

For example, as shown in FIG. 26, a latex sleeve 113 may be the securing means used to attach the catheter 102 to the endoscope 10 (or more particularly, the first channel 101). The sleeve 113 may be fitted to extend along a length (partial or full length) of the catheter 102.

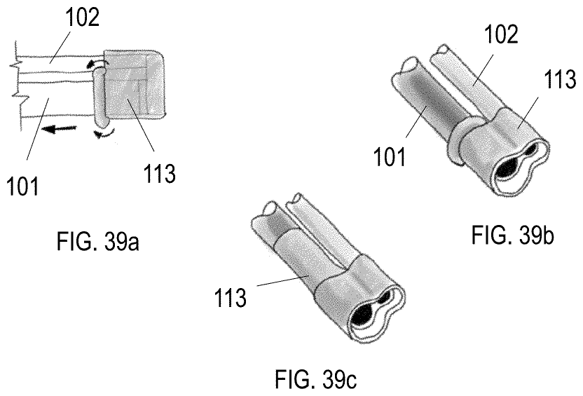

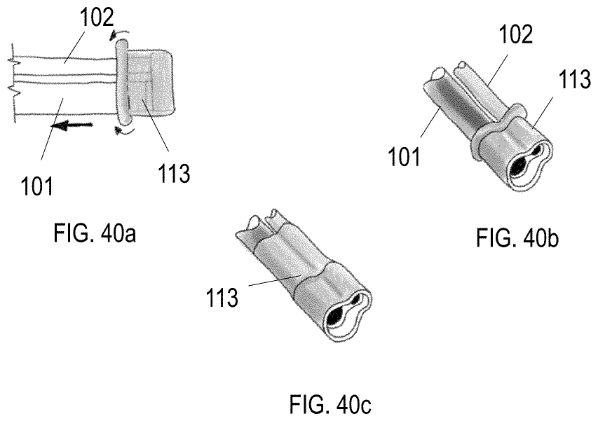

In some embodiments, for example, as shown in FIGS. 39a-c and FIGS. 40a-c, the sleeve 113 may be fitted or otherwise attached to the distal ends of both the first channel 101 and the catheter 102. In the exemplary embodiment of FIGS. 39a-c, portions of the sleeve 113 may extend in a proximal direction around either the first channel 101 and/or catheter 102. Additionally, or alternatively, as shown in the exemplary embodiment of FIGS. 40a-c, the sleeve 113 may extend in a proximal direction around both the first channel 101 and catheter 102. It should be appreciated that embodiments of the sleeve 113 may be used to cover other securing means that may be use for attaching the first channel 101 to the catheter 102.

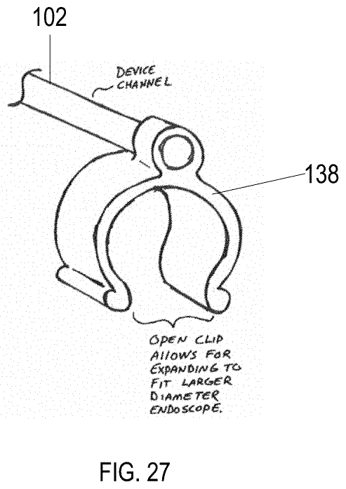

In some exemplary embodiments, as illustrated in FIG. 27, the means for securing the catheter 102 to the endoscope 10 may be achieved by an open clip 138 arranged at or near the distal end of the endoscopic device 100. It should be appreciated that the gapped portion of the open clip 138 allows for the open clip 138 to expand to fit (e.g., frictionally fit) larger diameter endoscopes 10.

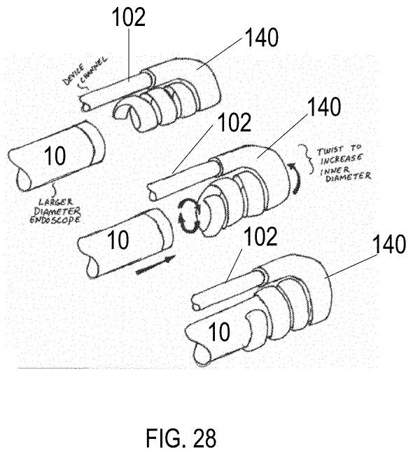

In some exemplary embodiments, as illustrated in FIG. 28, the means for securing the catheter 102 to the endoscope 10 may be achieved by a coiled or twisted cap 140 arranged at or near the distal end of the endoscopic device 100. In this embodiment, a twisting motion of the twisted cap 140 in one or both directions my cause the opening diameter of the twisted cap 140 to increase for receiving a larger diameter endoscope 10. For example, twisting the twisted cap 140 in one direction (opposite direction to a winding pitch) may result in a diameter of the twisted cap 140 to increase. When released from the twisting motion, the diameter of the twisted cap 140 may decrease to grip the endoscopic device 100 or endoscope 10. In some embodiments, the twisted cap 140 may be comprised of a thermoplastic; however, it should be appreciated that a wire (e.g., a metal wire and/or coated metal wire) may be used for forming the twisted cap 140.

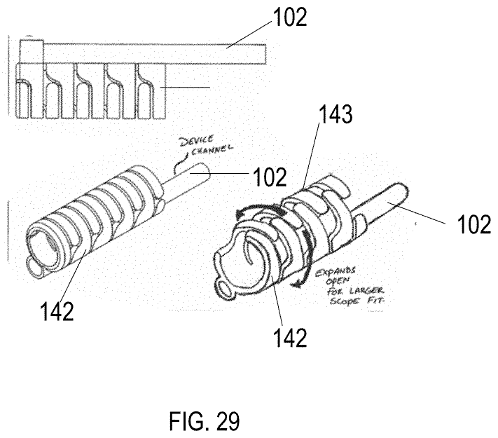

In some exemplary embodiments, as illustrated in FIG. 29, the means for securing the catheter 102 to the endoscope 10 may be achieved by an expandable clip 142 with curved movable leg portions 143 forming an opening in the expandable clip 142 for frictionally fitting the endoscope 10 at least partially therebetween. The curved leg portions 143 may extend from opposed sides of a clip body (not shown) to form the opening for the endoscope 10, and in some embodiments, may alternate such that no curved leg portions 143 extending from one side are next to each other when forming the opening. It should be appreciated that, in some embodiments, it may be desired to have leg portions 143 from one side next to each other when forming the opening. It should further be appreciated that the securing means may be formed of one or more elastomeric materials and/or thermoplastic materials.

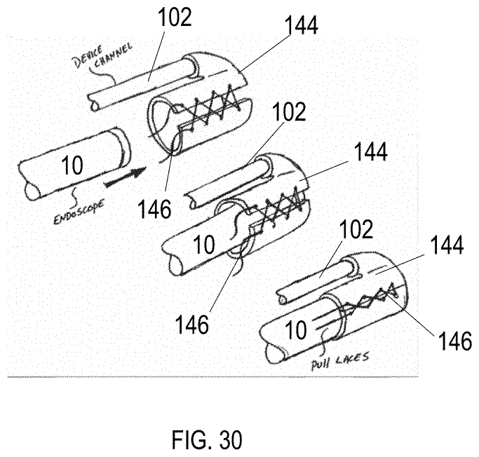

In some exemplary embodiments, as illustrated in FIG. 30, the means for securing the catheter 102 to the endoscope 10 may be achieved by a gapped clip 144 arranged at or near the distal end of the endoscopic device 100 and having one or more laces 146 for closing the gap formed in the gapped clip 144. The laces 146 may be secured or otherwise attached to the gapped clip 144 via one or more apertures or similar openings formed on opposed sides of the gap and in the side walls of the gapped clip 144. It should be appreciated that the laces 146 may be strung through the openings such that the lace 146 may be pulled in a direction (e.g., a proximal direction) to close the gap of the gapped clip 144 for attaching the endoscope therebetween.

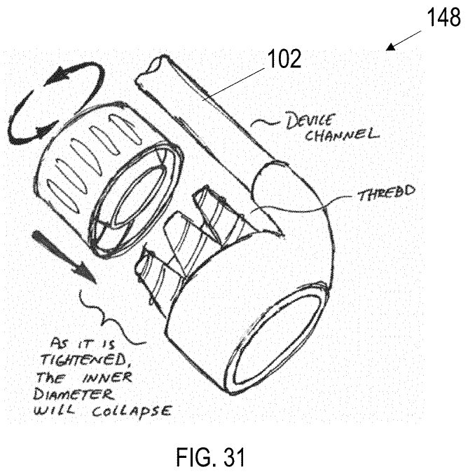

In some exemplary embodiments, as illustrated in FIG. 31, the means for securing the catheter 102 to the endoscope 10 may be achieved by a collapsible clip 148 arranged at or near the distal end of the endoscopic device 100. In this embodiment, for example, the distal end of the endoscope 10 may be frictionally fitted into an opening of the collapsible clip 148. It should be appreciated that frictionally fitting the endoscope 10 into the opening may cause an inner diameter of the collapsible clip 148 to collapse as the endoscope is tightened. For example, an adapter for the collapsible clip 148 may include a threaded portion that collapses the fingers of the collapsible clip 148 as it is threaded. In some embodiments, the adapter and/or fingers may be tapered for collapsing of the fingers to frictionally attach to a device.

In some embodiments, a mating piece may be provided with the collapsible clip 148. In this embodiment, the endoscope 10 may be attached to the mating piece and inserted or attached to the collapsible clip 148 for collapsing the inner diameter of the collapsible clip 148 to secure the endoscope 10. In some embodiment, the collapsible clip 148 may be a single piece construction that collapses upon securing (e.g., by rotation and/or press fitting) the distal end of the first channel 101 thereto.

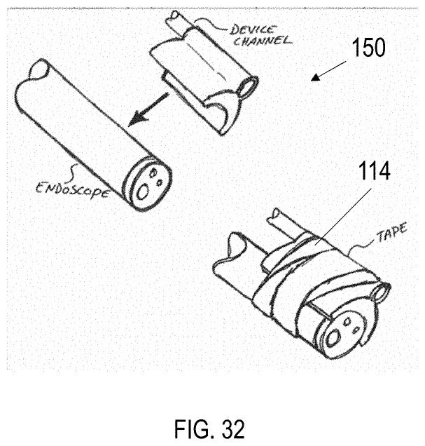

In yet another exemplary embodiments, as illustrated in FIG. 32, the means for securing the catheter 102 to the endoscope 10 may be achieved by a partial-clip 150 arranged at or near the distal end of the endoscopic device 100. In this embodiment, the partial-clip 150 may have an arcuate profile that corresponds to the shape and/or size of the endoscope 10. In some embodiments, for example, the partial-clip 150 may be secured or otherwise attached to the endoscope 10 using a tape 114 (FIG. 32) or similar fastening means.

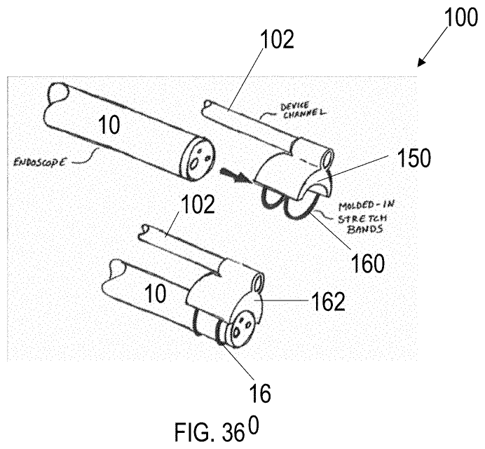

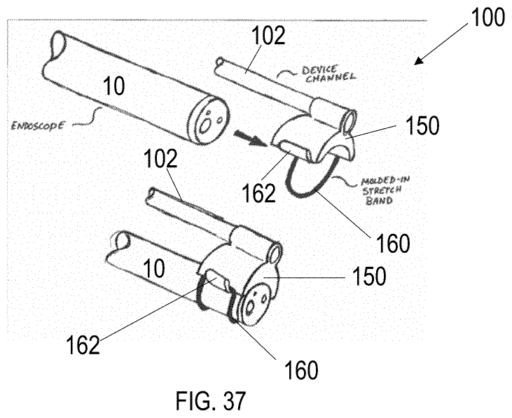

Additionally, or alternatively, the partial-clip 150 may be secured or otherwise attached to the endoscope 10 via one or more stretch (flexible) bands selectively attached to one or more sides (walls) of the partial-clip 150. In the exemplary embodiment of FIG. 36, stretch band(s) 160 are shown secured to both side walls of the partial-clip 150. In the exemplary embodiment of FIG. 37, the stretch band 160 is secured to one side wall of the partial-clip 150.

In some embodiments, for example, where the stretch band 160 is secured to one side wall, the partial-clip 150 may include a shoulder 162 extending outwardly from an outer surface of an opposite side wall of the partial-clip 150 for securing or otherwise attaching the stretch band 160 thereto and for securing the partial-clip 150 to the endoscope 10 (first channel 101).

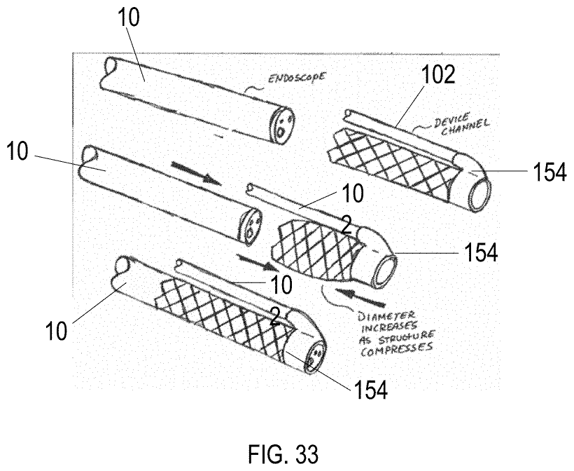

In some exemplary embodiments, as illustrated in FIG. 33, the means for securing the catheter 102 to the endoscope 10 may be achieved by a compressible clip 154 arranged at or near the distal end of the endoscopic device 100. In this embodiment, for example, as the endoscope 10 is inserted (frictionally fitted) into an opening in the compressible clip 154, the compressible clip 154 may expand to receive the endoscope 10 as it collapses around the endoscope 10 to secure the endoscope 10 to the compressible clip 154.

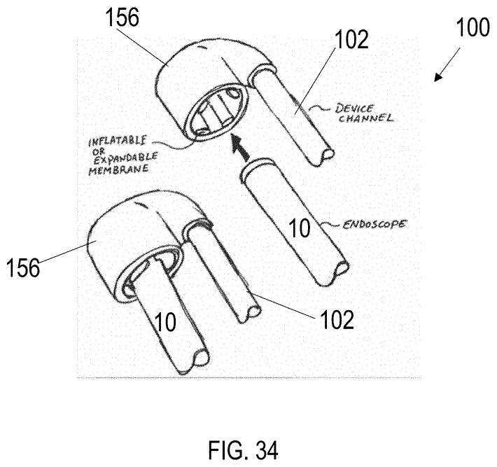

In some exemplary embodiments, as illustrated in FIG. 34, the means for securing the catheter 102 to the endoscope 10 may be achieved by an inflatable or expandable membrane 156 arranged at, near and/or inside a diameter of the distal end of the endoscopic device 100. In some embodiments, the membrane 156 may be formed from one or more of a neoprene, polyurethane, thermoplastic elastomer, polyisoprene, and/or silicone material. However, it should be appreciated that different materials known in the art for forming an inflatable or expandable membrane may be used.

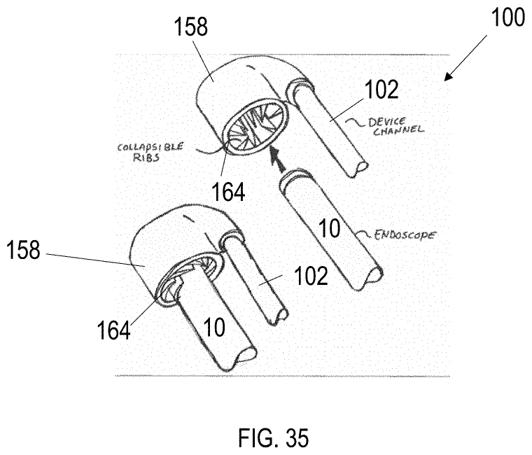

In yet a further exemplary embodiment, as illustrated in FIG. 35, the means for securing the catheter 102 to the endoscope 10 may be achieved by a ribbed clip 158 arranged at or near the distal end of the endoscopic device 100. The ribbed clip 158 may include one or more collapsible inner ribs 164 within an opening of the ribbed clip 158 for receiving the endoscope 10 at least partially therebetween. In this embodiment, as the endoscope 10 is inserted (friction fitted) into the ribbed clip 158, one or more of the inner ribs 164 may collapse around the endoscope 10 to secure or otherwise attach the endoscope 10 to the ribbed clip 158.

It should be appreciated that one or more of the securing means illustrated in FIGS. 27-40 includes an opening for at least partially securing or otherwise attaching one or more catheters 102 therebetween. The catheter 102 openings may be integral with one or more of the securing means described herein or be a separate part selectively attached to the securing means for securing or otherwise attaching the catheter 102 to the endoscope 10 at the distal end of the endoscopic device 100.

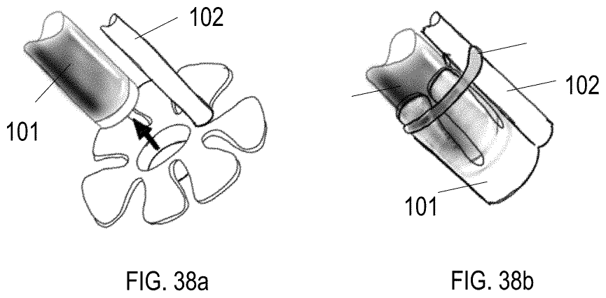

In some embodiments, the securing means may be a flower-like device (FIG. 38) with one or more collapsible or foldable side petals for wrapping around the first channel 101. In this embodiment, an additional piece (e.g., a tape 114 or similar securing band) may be used for securing the foldable sides around the first channel 101.

In some embodiments, the endoscope 10 and/or the catheter 102 may be coated with a lubricious coating. It should be appreciated that providing a coating (e.g., a lubricious coating) may improve endoscope delivery and/or motion during the endoscopic procedure. In some embodiments, the lubricious coating may be applied to the catheter 102 and/or a sleeve covering the catheter 102. Additionally, or alternatively, the lubricous coating may be beneficial inside the distal cap 112 to help with attachment. It should be further appreciated that external coats may be desirable for freely moving through anatomy. Internal coats (e.g., via a Teflon liner) may allow for low-friction passage of devices, for example, via the catheter 102.

It is to be understood that the detailed description is intended to be illustrative, and not limiting to the embodiments described. Other embodiments will be apparent to those of skill in the art upon reading and understanding the above description. Moreover, in some instances, elements described with one embodiment may be readily adapted for use with other embodiments. Therefore, any products, methods and/or systems described herein are not limited to the specific details, the representative embodiments, and/or the illustrative examples shown and described. Accordingly, departures may be made from such details without departing from the spirit or scope of the general aspects of the present disclosure.

Additionally, the components and materials described hereinafter as making up the various embodiments are intended to be illustrative and not restrictive. It should be appreciated that many suitable components and materials that would perform the same or a similar function as the materials described herein are intended to be embraced within the scope of embodiments of the present disclosure.

We claim:

1. An endoscopic device comprising:
a catheter having a distal end and a proximal end;
a securing means attached at the distal end of the catheter for securing an endoscope at the distal end; and
a mounting means attached at the proximal end for securing the endoscope at the proximal end.

2. The endoscopic device of claim 1, wherein the securing means includes a first opening for receiving the endoscope therebetween and a second opening for receiving the catheter therebetween.

3. The endoscopic device of claim 2, wherein the securing means is a sleeve.

4. The endoscopic device of claim 3, wherein the sleeve has an arcuate profile.

5. The endoscopic device of claim 3, wherein the sleeve is at least partially cylindrical.

6. The endoscopic device of claim 2, wherein the securing means includes a third opening at or near its distal end for leverage and/or angulation.

7. The endoscopic device of claim 1, wherein the securing means includes an opening arranged at the distal end to allow for an accessory to articulate.

8. The endoscopic device of claim 1 wherein the securing means includes an opening at the distal end arranged to provide an exit angle for an accessory traveling via the catheter, and wherein the securing means is arranged at the distal end such that the exit angle of the accessory differs from an axis of the endoscope.

9. The endoscopic device of claim 1, wherein the catheter is a feed catheter.

10. The endoscopic device of claim 2, further comprising a flap arranged at the distal end of the sleeve for covering one or more of the first opening or second opening.

11. The endoscopic device of claim 1, wherein one or more surfaces that interface with the endoscope, an exterior of the endoscopic device, or interior of the catheter includes a lubricious treatment.

12. The endoscopic device of claim 1 further comprising a balloon arranged at the securing means of the endoscopic device.

13. The endoscopic device of claim 12 further comprising an air feed channel operably attached to the balloon for inflating and deflating the balloon.

14. The endoscopic device of claim 12, wherein the balloon includes one or more fingers extending outwardly therefrom for engaging tissue.

15. The endoscopic device of claim 12, wherein the balloon surrounds portions of either or both the endoscope and the securing means.

16. The endoscopic device of claim 12, wherein the balloon is arranged to articulate an exit angle of an accessory exiting the catheter.

17. The endoscopic device of claim 1, wherein the catheter further includes a trap door at the distal end.

18. The endoscopic device of claim 1, wherein the endoscope is a dual channel endoscope.

19. The endoscopic device of claim 1 further comprising a balloon arranged within the catheter.

20. The endoscopic device of claim 19, wherein the balloon is arranged at the distal end of the catheter.
